(19) United States
(12) Patent Application Publication
(10) Pub. No.: US 2025/0308668 A1
(43) Pub. Date: Oct. 2, 2025

(54) METHODS AND SYSTEMS FOR LIGAMENT RECONSTRUCTION

(71) Applicant: INTELLIJOINT SURGICAL INC., KITCHENER (CA)

(72) Inventor: ANDRE NOVOMIR HLADIO, WATERLOO (CA)

(21) Appl. No.: 18/616,402

(22) Filed: Mar. 26, 2024

(51) Int. Cl. G16H 20/40; A61B 34/00; A61B 34/20

(52) U.S. Cl. CPC G16H 20/40; A61B 34/20; A61B 34/25; A61B 2034/2055; A61B 2034/2065

(57) ABSTRACT

Methods and systems for ligament reconstruction provide navigational assistance localizing at least one desired tunnel aperture on surface of a bone of a joint. In an embodiment, each of: a kinematic range of motion of the joint, a first tunnel aperture point for a first bone and a ligament graft; and a plurality of candidate second tunnel aperture points relative to a second bone; are registered. A plurality of datasets corresponding to the plurality of candidate second tunnel aperture points are defined, where the datasets represent the relationship of either or both of a graft length or a graft tension along the kinematic range of motion. A desired second tunnel aperture point is determined in response to a selection of a desired dataset. A user interface is provided to guide a probe to a target on the bone for the desired tunnel aperture.

### FIELD OF INVENTION

This application relates to computer assisted surgical procedures including methods and systems for ligament reconstruction, for example, anterior cruciate ligament (ACL) reconstruction.

### BACKGROUND

A surgeon may use a navigation system to assist in a surgical procedure. An optical sensor (e.g. an image sensor such as a camera) collects images of optically detectable trackers which are rigidly coupled to the patient's anatomy, to surgical instruments, and, as may be applicable, implant components.

Once registration is complete (as may be applicable), the navigation system continuously estimates the position and location of the trackers and the objects to which they are attached by determining the pose of the trackers from the images. Using the relative locations and positions of trackers attached to a patient's bone, a navigation system can provide accurate measurements to assist the surgeon with the procedure such as a joint reconstruction procedure.

In a ligament graft procedure, respective ligament ends are fixed to respective bones of a joint to be reconstructed e.g. at native attachment areas of such bones. In various procedures, such as anatomic single-bundle ACL reconstruction (ASB-ACLR) procedures, femoral and tibial tunnel apertures are created at different locations within the native ACL attachment area. Each tunnel aperture represents a point of attachment of the ligament graft to the respective bone, with the graft extending between an selected pairs of apertures, one of the femur and one of the tibia. Graft length changes are discussed in Tanabe et al, “Comparison of Graft Length Changes During Knee Motion Among 5 Different Anatomic Single-Bundle Anterior Cruciate Ligament Reconstruction Approaches: A Biomechanical Study” Orthop J Sports Med. 2019 Mar. 26; 7 (3): 2325967119834933; doi: 10.1177/2325967119834933, (hereinafter “Tanabe”), the entire contents of which are incorporated herein by reference. As shown in Tanabe, Graft length changes during knee motion will be different among procedures with different tunnel aperture locations.

Two known methods to evaluate ACL graft isometry during knee motion comprise an assessment of 1) changes in graft length, or 2) changes in graft tension. As further noted in Tanabe at p.2, (references removed), “Biomechanical studies have shown a statistically significant correlation between graft length versus the knee flexion angle curve and also between graft tension versus the knee flexion angle curve in each graft. In addition, a statistically significant correlation has been found between the maximum value in the length changes and the maximum value in the tension changes.”

It is therefore desired to assist with placement of a ligament graft, such as the ACL graft, for example, to recreate a desired strain curve throughout a range of motion.

### SUMMARY

Methods and systems for ligament reconstruction provide navigational assistance localizing at least one desired tunnel aperture on surface of a bone of a joint. In an embodiment, each of: a kinematic range of motion of the joint, a first tunnel aperture point for a first bone and a ligament graft; and a plurality of candidate second tunnel aperture points relative to a second bone; are registered in the system. A plurality of datasets corresponding to the plurality of candidate second tunnel aperture points are defined, where the datasets represent the relationship of either or both of a graft length or a graft tension along the kinematic range of motion. A desired second tunnel aperture point is determined in response to a selection of a desired datasets. A user interface is provided to guide a probe to a target on the bone for the desired tunnel aperture.

Features and aspects will be appreciated, from the embodiments shown and described, such as those set forth in the following numbered statements:

Statement 1: A navigation system for ligament reconstruction comprising: a probe with a tip, a tip location of the tip trackable by the navigation system; a computing device coupled to the navigation system, the computing device comprising at least one processor and memory storing computer-readable instructions executable by the at least one processor to cause the computing device to: register a kinematic range of motion of a joint, the joint comprising a first bone and a second bone; register a first tunnel aperture point for a first bone and a ligament graft in response to the tip location as tracked; register a plurality of candidate second tunnel aperture points relative to the second bone in response to respective tip locations as tracked; define a plurality of datasets corresponding to the plurality of second tunnel aperture points, wherein the datasets represent the relationship of either or both of a graft length and a graft tension throughout the kinematic range of motion of the joint; select a desired dataset from the plurality of datasets; and determine a desired second tunnel aperture point on the second bone based on the desired datasets.

Statement 2: The system of Statement 1, wherein the joint is a knee, the first bone is a tibia, the second bone is a femur and the ligament is an anterior cruciate ligament (ACL).

Statement 3: The system of Statement 1, wherein the navigation system further comprises one or more of: an optical camera configured for providing optical information for tracking objects, the objects comprising the first bone, the second bone and the probe; a first bone tracker configured for coupling to the first bone for tracking the first bone; or a second bone tracker configured for coupling to the second bone for tracking the second bone.

Statement 4: The system of Statement 3, wherein the kinematic range of motion is based on poses of the first bone tracker and/or the second bone tracker during a range of motion.

Statement 5: The system of Statement 1, wherein the computer-readable instructions are executable by the at least one processor to cause the computing device to provide a user interface to display the plurality of datasets.

Statement 6: The system of Statement 5, wherein to select the desired dataset comprises receiving user input to select one of the datasets as the desired dataset.

Statement 7: The system of Statement 1, wherein the computer-readable instructions are executable by the at least one processor to cause the computing device to provide a user interface to display the plurality of datasets as respective curves.

Statement 8: The system of Statement 1, wherein the computer-readable instructions are executable by the at least one processor to select the desired dataset automatically without user selection.

Statement 9: The system of Statement 1, wherein the computer-readable instructions are executable by the at least one processor to cause the computing device to provide a user interface to guide a user to identify the desired second tunnel aperture point on the second bone using the tip of the probe.

Statement 10: The system of Statement 9, wherein the computer-readable instructions are executable by the at least one processor to cause the computing device to generate a surface model associated to the plurality of candidate second tunnel aperture points, and wherein the user interface is configured to display the probe tip as tracked and the desired second tunnel aperture point with respect to the surface model.

Statement 11: The system of Statement 1, wherein to register the first tunnel aperture point defines a spatial relationship between the first tunnel aperture point identified by the tip of the probe and a pose of a first tracker coupled to the first bone, and wherein the pose of the first tunnel aperture point is determinable from tracking the first tracker.

Statement 12: The system of Statement 1, wherein the computer-readable instructions are executable by the at least one processor to cause the computing device to register the first tunnel aperture point as the computing device registers the kinematic range of motion of the joint, the computing device tracking each of the probe tip and the second bone during the range of motion.

Statement 13: The system of Statement 1, wherein the computer-readable instructions are executable by the at least one processor to cause the computing device to register the plurality of candidate second tunnel aperture points by receiving the respective tip locations as either: A) points marking an area of the second bone or B) a painting of a surface patch of the second bone; the computer-readable instructions executable by the at least one processor to cause the computing device to determine the candidate second tunnel aperture points within the area or surface patch. The system of claim 1, wherein the navigation system further comprises or is communicatively coupled to a robotic system, and wherein the computer-readable instructions are executable by the at least one processor to cause the computing device to align a trajectory guide of the robotic system with the desired second aperture point.

Statement 14: A computer-implemented method comprising: registering a kinematic range of motion of a joint, the joint comprising a first bone and a second bone; registering a first tunnel aperture point for a first bone and a ligament graft in response to a tip location of a probe as tracked; registering a plurality of candidate second tunnel aperture points relative to the second bone in response to respective tip locations of the probe as tracked; defining a plurality of datasets corresponding to the plurality of second tunnel aperture points, wherein the datasets represent the relationship of either or both of a graft length and a graft tension throughout the kinematic range of motion of the joint; selecting a desired dataset from the plurality of datasets; and determining a desired second tunnel aperture point on the second bone based on the desired dataset.

Statement 15: A method to perform a ligament reconstruction comprising: registering to a navigation system a kinematic range of motion of a joint comprising a first bone and a second bone; registering to the navigation system a first tunnel aperture point for the first bone and a ligament graft using a probe having a tip trackable by the navigation system; registering to the navigation system a plurality of candidate second tunnel aperture points relative to the second bone using the probe as tracked; selecting a desired datasets from a plurality of curves representing the relationship of either or both of a graft length and a graft tension throughout the kinematic range of motion of the joint, wherein the plurality of datasets are presented by a user interface; and identifying a desired second tunnel aperture point on the second bone based on the desired dataset as selected.

Statement 16: The method of Statement 15, wherein the navigation system is configured to track the first bone and the second bone using an optical sensor comprising a camera.

Statement 17: The method of Statement 15, wherein the joint is a knee, the ligament graft is an anterior cruciate ligament (ACL), the first bone is a tibia, and the second bone is a femur.

Statement 18: The method of Statement 15, further comprising receiving guidance from the navigation system for locating, using the tip of the probe, the desired second tunnel aperture point on the second bone.

Statement 19: A computer-implemented method comprising:
displaying a plurality of datasets representing the relationship of either or both of a graft length and a graft tension throughout a kinematic range of motion of a joint comprising a first bone and a second bone; receiving input selecting a desired dataset from the plurality of datasets; and displaying a target location of a desired second tunnel aperture point associated with the second bone, along with a display of a current location of a probe tip, responsive to the actual position of a probe of a navigation system and in a common coordinate frame to the desired second aperture point.

Statement 20: The method of Statement 19, wherein each of the plurality of datasets is associated with one of a plurality of second tunnel aperture points on the second bone, and wherein the desired second tunnel aperture point is determined in accordance with the association.

Additional aspects include computer program products. Such products may comprise a non-transient storage device, such as memory, storing computer-readable instructions executable by a processor (which may be one or more processors) to cause a computing device (which may be one or more such devices) to perform a computer implement method.

### BRIEF DESCRIPTION OF THE DRAWINGS

FIG. 1 is an overview of an exemplary intraoperative navigation system for an ACL reconstruction procedure, in accordance with an embodiment, for example, where a computing device, such as a laptop, is configured as disclosed herein.

FIG. 2 is an illustration of a knee joint showing a first tunnel aperture point and various candidate second tunnel aperture points in accordance with an embodiment.

FIGS. 3A and 3B are representations of knee joint in a first position and a second position, where the knee joint is annotated with a first tunnel aperture point on a tibia and four (4) candidate second tunnel aperture points on a femur.

FIGS. 4A and 4B are respective flowcharts, where FIG. 4A shows operations of a computer implemented method and FIG. 4B shows steps of a complimentary user method, each in accordance with an embodiment.

FIG. 5 is an illustration of a user interface, in accordance with an embodiment, displaying a graph of changes in graft length for various pairings of tunnel aperture points.

FIG. 6 is an illustration of a user interface, in accordance with an embodiment, displaying a graphical device to guide placement of a desired second tunnel aperture point.

FIG. 7 is a flowchart showing operations of a computer implemented method in accordance with an embodiment.

It will be appreciated that for simplicity and clarity of illustration, elements shown in the figured have not necessarily been drawn to scale. For example, the dimensions of some of the elements may be exaggerated relative to other elements for clarity.

### DETAILED DESCRIPTION

Described herein are methods and systems for performing a navigated surgical procedure involving a patient's anatomy. The primary example disclosed herein is a navigation-assisted ACL reconstruction. However, it should be evident that the systems, devices, apparatuses, methods and computer-implemented methods described herein may be applied to other ligaments and other joints requiring treatment where assessment through a range of motion is desired. For example, such ligaments and/or joints may comprise other knee ligaments of knee joints, elbow ligaments of elbow joints, and ankle ligaments of ankle joints.

FIG. 1 illustrates an exemplary intra-operative navigation system 100, in the context of a navigation-assisted ACL reconstruction. In this intra-operative navigation system 100, an image sensor 102 is shown located on a moveable cart 104, with its field of view oriented towards a surgical site 106. The image sensor 102 could alternatively be mounted on an anatomic structure of the patient (e.g. a bone such as the femur), held in the hands of the operator, worn by an operator (e.g. as a head mounted device), coupled to a mounting arm or structure or any other appropriate position. Image sensor 102 comprises one or more sensor devices, for example, a camera for determining image sensor data. Other sensors of device 102 may comprise devices for determining directional information such an accelerometer, gyroscope, etc.

One or more trackers may be attached to various objects, including an anatomic structure (typically to a hard structure thereof that is not susceptible to deformation, such as a bone) of a patient and/or a surgical instrument. The one or more trackers provide optically detectable features for detection by the image sensor 102. In the embodiment shown in FIG. 1, a first tracker 108 is coupled to an anatomic structure of a patient (i.e. a tibia 110), a second tracker 112 is attached to a femur 114 and a third tracker 116 is coupled to a surgical instrument 118. Instrument 118 in this case is a probe (e.g. a tracked probed) having a probe tip 120.

While a single optical sensor 102 is shown in FIG. 1, more than one can be employed. When an optical sensor is located on a bone, for example, (and registered in system 100), a separate tracker device for the bone need not be used, as will be apparent to a person of skill in the art.

The skilled person will understand that there can be any number of trackers coupled to any number of anatomic structures and/or surgical instruments. The skilled person will understand that there can be fewer instances of distinct trackers. For example, in accordance with the surgical workflow, a first tracker may be coupled to a first object for a portion of the workflow and at a different time, the first tracker may be coupled to a second object for a different portion of the workflow.

Where the navigation system 100 comprises two or more trackers, the trackers may be identical to each other. In an embodiment, the two or more trackers may have different optically detectable features such that the navigation system can differentiate the trackers. For instance, the trackers may have different colors, geometries, sizes, or numbers and/or arrangement of optically detectable features (e.g. retro-reflective spheres as shown in FIG. 1).

Image sensor 102 transmits image sensor data (including image data or pose data associated with the trackers, such as trackers 108, 112 and/or 116) to a computing device 122. Image sensor 102 may be communicatively coupled to computing device 122 by wire (as shown). Alternatively, communication between image sensor 102 and computing device 122 may be wireless communication. The computing device 122 may comprise a laptop, workstation, or other computing device having at least one processing unit and at least one storage device such as memory storing software (instructions and/or data) as further described herein to configure the execution of the computing device such as to perform operations of a method. System 100 may comprise one or more computing devices. A computing device may comprise a cloud server and/or remote computing devices.

Computing device 122 performs applicable processing to calculate the poses of one or more trackers. Where the trackers have a known spatial or geometrical relationship to a coupled object, such as via registration, computing device 122 also performs the applicable processing to calculate the pose of the coupled objects. For example, where a tracker, such as tracker 108, is coupled and registered to the anatomic structure (e.g. tibia 110, where another anatomic structure comprises femur 114, respectively a first bone and a second bone of a joint of a patient), the pose of the anatomic structure may be determined by computing device 122. Further, where a tracker, such as tracker 116, is coupled to a surgical instrument, such as surgical instrument 118, computing device 122 may determine the pose of the surgical instrument using the known spatial relationship between the tracker and the surgical instrument. Computing device 122 may further determine a relative pose between two or more objects, such as between the first bone and second bone, a surgical instrument and an anatomic structure of a patient's anatomy 116 (e.g. femur or tibia), etc. Any pose may be determined in three dimensions, and comprises position, location and/or orientation of an object. The computing device 122 may further display clinically relevant information to the user, including tracking information, wherein tracking information may comprise image data and/or pose data associated with the pose of one or more trackers and one or more objects to which the trackers are coupled. For example, tracking information may comprise image data and/or pose data associated with trackers 108, 112 and/or 116 and the objects to which they are coupled, such as surgical instrument 118, and an anatomic structure of a patient's anatomy, respectively. Other clinically relevant information may comprise measurements determined from, for example, poses of the objects. Measurement information may include (relative) distances, (relative angles), planes, axes, etc. determined using known techniques. For example, a distance (e.g. a Euclidean distance) may be determined between a point indicated on a tibia and a point indicated on a femur.

In order to determine the pose of probe tip 120, the geometry of tracker 116 relative to probe tip 120 is known to the system (i.e. to computing device 122). The geometry can comprise the relationship between tracker 116 and the probe (e.g. a direction and distance of the probe tip from the tracker) This can be achieved by designing and manufacturing probe tip 120 and tracker 116 as separate components that can only be assembled in a single unique configuration, such as a trackable probe. In an embodiment, the trackable probe may be designed and manufactured as a single integral component. In another embodiment, registration or calibration steps can be performed to determine the spatial relationships between probe tip 120 and tracker 116.

In use, such as in a navigation-assisted procedure, intra-operative navigation system 100 is registered to the patient's anatomy; that is, the positional and geometric relationships between at least some of the patient's anatomic planes/axes/features/landmarks are known to computing device 114. It is understood that the camera and objects are registered to surgical navigation system 100 in accordance with a registration procedure or procedures. For example, a method and system for surgical navigation is disclosed in Applicant's U.S. Patent U.S. Pat. No. 9,247,998 B2, granted Feb. 2, 2016, and entitled “System and Method of Intra-Operative Leg Position Measurement”, the content of which is incorporated herein by reference in its entirety.

It is desired to determine graft length measurements through a range of motion of a joint, for example, to determine curves for candidate graft locations. In an embodiment, components of system 100 can be used to register (in system 100) a plurality of tunnel aperture points on joint surfaces. The points represent candidate tunnel aperture points for the reconstruction procedure. The computing device can be configured to track (e.g. capture) information from optical sensor 102 as the joint is moved through the range of motion and perform determinations of relative distances such between a first tunnel aperture on a first bone and respective second candidate tunnel aperture points on a second bone. Workflow including information provided to a user in a user interface can guide the operations (e.g. steps of a method) to perform a range of motion tracking and to register points for determining the graft length measurements.

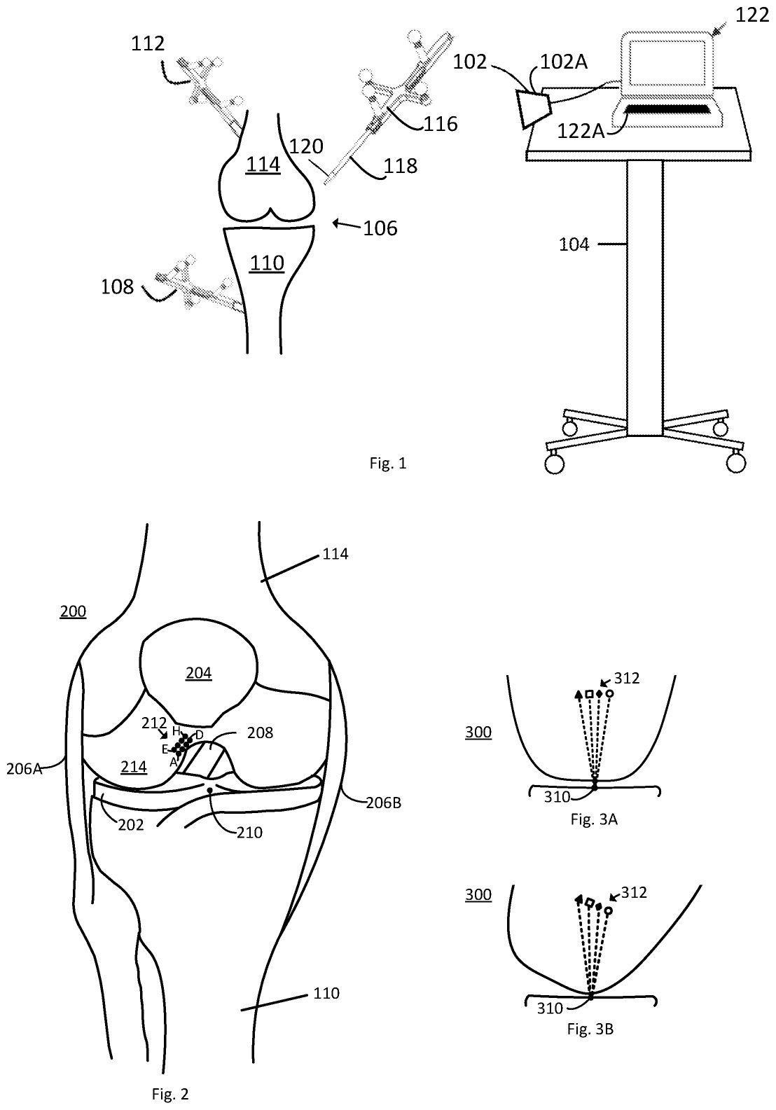

FIG. 2 is an illustration of an exposed knee joint 200 comprising tibia 110 and femur 114. Also shown are additional structures such as a meniscus 202, a patella 204, collateral ligaments 206A, 206B and a posterior cruciate ligament (PCL) 208. A native ACL is removed and not shown. The joint is shown for illustrative purposes and is not intended to suggest a grossly invasive approach to an ACL reconstruction.

FIG. 2 shows a first tunnel aperture point 210 on tibia 110 and a plurality (e.g. 8) of candidate second tunnel aperture points 212 on a lateral condyle 214 of femur 114. The candidate second tunnel aperture points 212 comprise a first row e.g. A to D and a second row E to H, though only some are labelled for clarity).

Further, for brevity, only a single first tunnel aperture point 210 is shown and described but a plurality of such points may be used and system 100 configured accordingly. In an embodiment, first tunnel aperture point 210 represents an actual tunnel aperture formed in tibia 110 during a procedure (steps not shown). Alternatively, the point 210 can represent a candidate point, such as one of a plurality of first candidate points.

In FIG. 2 and for purposes of illustration, the positions of the first tunnel aperture point 210 and candidate second tunnel aperture points 212 are representational in that such positions may not correspond to actual desired positions of any particular preferred ACL reconstruction procedure. The points are relatively large in scale for illustration purposes only. FIGS. 3A and 3B are representations of knee joint 300 (e.g. a partial medial view of the lateral condyle) in a first position (e.g. full extension) and a second position (partial flexion), where the knee joint 300 is annotated with a first tunnel aperture point 310 on a tibia and 4 candidate second tunnel aperture points 312 on a femur. It is apparent from a visual comparison of FIGS. 3A and 3B that the relative lengths between the first tunnel aperture point 310 and the candidate second tunnel aperture points 312 change with rotation and respective lengths for different pairs have different changes.

System 100 can be configured to determine graft length measurements between any of candidate second tunnel aperture points 212 and first tunnel aperture point 210 such as in respective pairs. An example is B to 210, or H to 210, etc. The measurements can be determined with the joint in any position such as through a range of motion. The range may be between a full extension position and a full flexion position (e.g. of) 120°, for example. Measurements at different range of motion waypoints (e.g. at spaced waypoints) between full extension and full flexion (along the range of motion) can be used to determine a curve for the candidate point pairing or pairings.

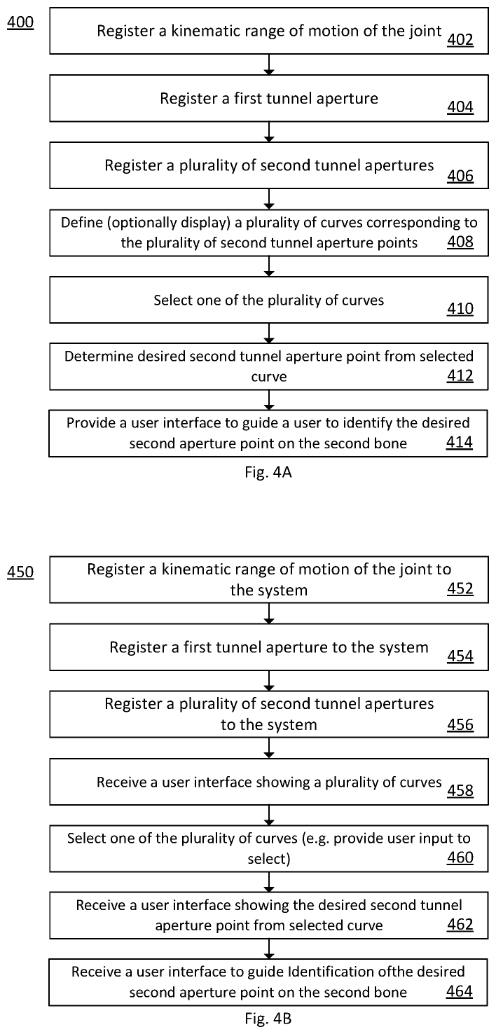

FIG. 4A is a flowchart of operations 400 of a computer-implemented method in accordance with an embodiment. FIG. 4B is a flowchart of steps of a user method 450 in accordance with an embodiment, for example, complimentary to operations of the computer-implemented method 400.

At operation 402, after optionally registering the tibia and femur (steps not shown), a user manipulates the joint through a range of motion (e.g. varying knee flexion angle) and the computing device registers the kinematic range of motion of the joint. With the trackers 108 and 112 in the field of view of optical sensor 102, computing device tracks the first bone (e.g. tibia 110) and the second bone (femur 114). The poses of the trackers 108, 112 can be determined from respective optical information to provide relative positions of the two bones. With two trackers, either bone may move during a tracking. User operation 452 comprise registering the kinematic range of motion of the joint to system 100. An anatomical registration of the patient's anatomy (e.g. respective joint bones, etc.) is not necessary. That is, tracking the poses of the trackers to determine changes between different captures does not need to be related to any coordinate frame for the patient's anatomical structure. The poses of the trackers in the optical information is sufficient for purposes of capturing a range of motion. Further alternative embodiments to tracking and capturing range of motion (e.g. using only a single dedicated bone tracker and a probe) are provided herein below.

In an embodiment, the range of motion corresponds to the joint moving from a first position to a final position with waypoints therebetween as previously described. For example, a first position comprises a full extension or 0° flexion and a final “full flexion” position comprises a flexion at e.g. 120°. Waypoints may be at 15°, 30°, 60° and 90° of flexion. Other (e.g. clinically relevant) positions or spacing may be used. More or fewer may be used.

In an embodiment for discrete captures at predefined points of a range of motion, user input is provided via an input device and the input is correspondingly received by computing device 122 to indicate to the computing device when to capture the optical information from the sensor 102 for the registration of poses, which are then used for length determinations. The input device can comprise a button 102A on optical sensor 102, or a key of a keyboard 122A of device 122, a pointing device (not shown), a touch enabled screen (not shown), a foot pedal (not shown), or other input device including a microphone such as for a voice enable interface. The user manipulating the joint may be different from the one operating the input device.

In an embodiment, computing device 122 is provided with workflow for the operations 402 including presentation(s) via a display device and/or speaker of information to prompt the range of motion operations. Feedback can be provided from system 100 to a user (e.g. visually, aurally, etc.) to confirm the registration e.g. as each position of the range of motion is registered. Workflow can indicate whether all or some requires repeating (e.g. a “do over”) to capture information.

In an embodiment, the range of motion is performed more than one time (example, three times) and poses are determined for each instance of the range. The respective poses at respective flexions positions are combined, for example, to improve data quality.

In an embodiment (which may be performed with any of the data gathering operations described herein for the range of motion), different types of kinematic range of motion operations may be performed. For example, a user may perform a range of motion (“ROM”) operation with different stresses on the joint. Types may comprise: a “normal” ROM, a “varus stress” ROM, a “valgus stress” ROM, or an “anterior stress” RoM. A user can apply stress during the range of motion by manually applying pressure on the knee as the joint is articulated. Any type of ROM operation may be repeated (e.g. 3 times) such as previously described.

A similar procedure is commonly done to assess soft tissues in knee procedures. The stressed RoMs provide for different knee kinematics. Poses for each type can be recorded (and multiple instances combined such as when a particular ROM is repeated to improve data quality) by computing system 122. Respective graft strain curves can be determined.

In an alternative embodiment to discrete captures at specific points in the range of motion triggered such as by user input, the range of motion capture is performed in a continuous fashion. For example, the range of motion is performed in association with a start input that indicates the first position (e.g. at extension (i.e. 0° flexion)) and a stop input that indicates a final position of the range of motion. The computing device records pose pairs for each of the bones of the joint for the time period during the time between the start and stop input, for example, sampling such poses using a sample period. The computing device 122 can be configured to determine a relative angle of flexion for a pose pair using the relative distance or change thereof and the first pose pair as a baseline, for example, indicating starting from no flexion. The pose pairs for predetermined flexion points (e.g. at desired degrees of flexion) may be selected from the sampled pairs. Further alternative embodiments are described herein below.

In an further alternative that operates in a continuous fashion, the start and stop inputs (or system driven prompts to a user) indicate the time period during which the range of motion is conducted, however, the joint need not start at a predefined position and end at a predefined position. Computing device 122 effectively records the pose pairs during the time period, determines relative angles for pairs, which may be changes in angle measured from a baseline pair, and orders the data set to determine a change of length, tension or both through the range of motion. Each pose pair may be a candidate baseline. Computing device 122 determines changes in angle for each other pair and determine the desired baseline by evaluating the magnitudes of the changes in angle. For example, a candidate baseline in which all of the relative changes in angle are negative or which has a relative change in angle to another pair that is a maximum between all of the others may represent a pose pair at full extension.

In an embodiment, a separate tracking modality is employed, such as a video base knee ROM tracker. Further alternative tracking may comprise ultrasound based ROM tracking and fluoroscopy based ROM tracking. In any such an embodiment, the modality of tracking is co-registered to the navigation system 100 to enable appropriate mapping.

At operation 404, the computing device registers the first tunnel aperture (e.g. point 210) by tracking probe tip 120 of the tracked probe using tracker 116, and the tibia using tracker 108. Workflow can be configured to prompt the user to apply the probe tip to a desired location on tibia 110. With the trackers 108 and 116 in a field of view of optical sensor 102, the optical sensor receives information for poses of each and to register the pose of the probe tip as a specific point on the tibia trackable by tracker 108. User operations 454 comprise registering the first tunnel aperture to system 100. An alternative embodiment to register the first tunnel aperture point without need of tracker 108 is described further below.

A user input may be provided via an input device and that input is correspondingly received by computing device 122 to indicate to the computing device when to capture the optical information from the sensor 102 for such determinations. The input device can comprise the button 102A on optical sensor 102, the key of the keyboard 122A of device 122, the foot pedal (not shown), or other input device, e.g. all as previously described. The user operating the probe tip 120 may be different from the one operating the input device.

At operation 406, the computing device registers the plurality of candidate second tunnel apertures (e.g. points 210), tracking probe tip 120 of the tracked probe using tracker 116, and the femur using tracker 112. Workflow can be configured to obtain optical information for candidate second tunnel aperture points 212 using tip 120 and tracker 112 associated to femur 114, which may be responsive to various inputs via the input device to indicate when to obtain the poses. Workflow can be configured to determine a predetermined number of such points or an open number (e.g. prompting the user to add subsequent points until the user provides input that no additional point is desired). User operations 456 comprise registering the plurality of candidate second tunnel apertures to system 100.

Workflow can be configured to delete a determined first tunnel aperture point or second tunnel aperture point or to replace one of same, etc.

Though not shown, in an embodiment, a user interface may be presented (e.g. displayed on a display screen) that includes a “live” view of image(s) from the optical sensor 102. In an embodiment one or more images from the live view can be annotated, such as via an overlay to each annotated image to be displayed, with graphical representations of the points 210, 212 determined by computing device 122. Once registered in computing device 122, the locations of the candidate first and second aperture tunnel points (210, 212) track with the respective trackers 108 and 112. Respective overlays are determined to provide annotations that match with the locations of the points 210, 212 in the images.

Though not shown, in an embodiment computing device 122 can be configured to generate a surface model based on the plurality of candidate second aperture points. In an embodiment, the points (e.g. A to H) can define a point cloud in the 3D coordinate frame of system 100, each point representing a single spatial measurement on the object's surface. The user interface can be configured to display the surface model showing the aperture points.

In an embodiment, computing device 122 is configured to determine the distance between candidate first tunnel aperture point 210 and at least one point of candidate second tunnel aperture points 212 (e.g. is respective pairs such as (A, 210), (F, 210), etc. Because the points are registered to computing device 122, the computing device 122 can compute the locations of the point pairs in a same coordinate system and determine the distance between each point of a pair over the range of motion using the stored pose pairs. A change of graft length can then be determined relative to a baseline length for example. In an embodiment, computing device 122 can compute locations for all available pairs. Each pairing of the first tunnel aperture point 210 with a respective candidate second tunnel aperture point (e.g. A to H) has a respective dataset (e.g. each set comprising values for multiple points of the range of motion) for any or all of graft length, change of length, graft tension or change of graft tension. The stored data may comprise only graft length with device 122 configured to compute other information therefrom as/when needed. Tension may be determined using a function that relates tension (force) and length, such as a linear spring equation where force=K*distance, for example.

The dataset may be displayed in a user interface such as by display of text and/or graphical representation of such data. For example, but not to be limiting, the datasets for the candidates may be shown as a graphs of curves of changes in graft length vs the knee flexion angle.

At operation 408, in an embodiment, computing device 122 defines and displays a plurality of curves corresponding to at least some of the plurality of second tunnel aperture points. Each curve represents the relationship of either or both of a graft length and a graft tension throughout the kinematic range of motion of the joint. In an embodiment, computing device presents a selected curve type e.g. graft length or tension or both, such as in accordance with user input (e.g. toggle between types). User operation 458 comprises receiving a user interface showing a plurality of curves.

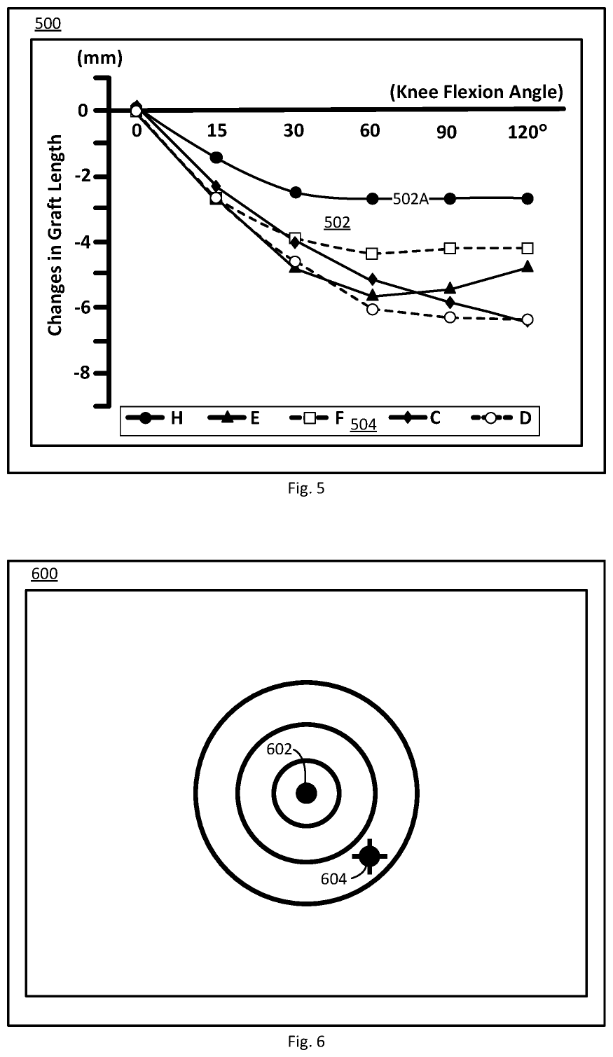

FIG. 5 is an illustration of a user interface 500, in accordance with an embodiment, displaying a graph having a plurality of curves 502, each (e.g. 502A) showing a change in graft length for various pairings of tunnel aperture points. The graph does not represent any particular clinical example and is illustrative only. Each curve (e.g. 502A) is relative to one of the candidate second tunnel aperture points (e.g. H, E, F, C or D) as shown in the legend 504. Only some curves from the total number of pairings are shown for clarity. Though shown as a two-axis plot, other representation(s) of the datasets may be shown in the alternative or in addition. For example, a radial plot may be determined for each pairing (not shown) using the degrees of the flexion. In an embodiment, datasets for pairs are displayed in a tabular form (not shown) (e.g. rows and columns). Rules can be applied to highlight particular data or minimize particular data in the table, for example, comparing data to one or more thresholds and highlighting some values or minimizing other values accordingly. Rules or other heuristics or model (e.g. an SVM (a support vector machine)) may be applied to identify one or more preferred datasets and the display thereof be responsive to such analysis.

At 410, operations select one of the curves (e.g. datasets). In an embodiment, selection is responsive to user input via an input device to choose one of the curves. In an embodiment, selection is performed using an SVM, a set of rules or heuristics, or other automated manner trained or defined to analyze the curves and select one from the set the satisfies various selection criteria, typically choosing an optimal curve from the set. The rules or heuristics can be responsive to change of length or tension thresholds, etc. User operations 460 comprise selecting one of the plurality of curves (e.g. providing user input to select one).

At 412, operations determine a desired second aperture point on the second bone (e.g. 114) based on the curve (dataset) as selected, optionally displaying the desired second aperture point in a user interface. The desired point is determined as the one of the candidate second tunnel aperture points that is associated to the curve. For purposes of the example herein, curve 502A is selected and is associated with second tunnel aperture point H. User operation 462 comprises receiving a user interface showing the desired second tunnel aperture point from the selected curve (UI not shown). In an embodiment, user interface 500 is updated to reflect the desired second aperture point, for example, highlighting the curve and/or the point displayed in legend 504, etc. Live view images may be annotated showing the desired second aperture point, for example, using a distinguishing color or graphic shape color or removing the other annotations for candidates not selected.

In an embodiment, such as shown at operation 414, system 100 (e.g. computing device 122) can be configured to provide a user interface to guide a user to identify the desired second aperture point H on the second bone (e.g. 114) using the probe tip 120. As the candidate points A to H are not physically marked on the lateral condyle 214, the location (e.g. determinable from the registered pose thereof) by may not be immediately apparent. The pose is stored (registered) in system 100 such that a pose of the tracked probe (e.g. tip 120) can be tracked and guided to the desired second aperture point H. User operation 464 comprises receiving a user interface to guide an identification of the desired second aperture point (e.g. H) on the second bone (e.g. femur 114).

FIG. 6 is an illustration of a user interface 600, in accordance with an embodiment, displaying a graphical device to guide placement of the desired second tunnel aperture point H. The graphical device comprises a bullseye where the location of the desired second aperture point H on the femur is represented by the center 602 of the bullseye's eye. The location is determined from the pose of the respective point (i.e. a previously determined spatial relationship between the point and the femur tracker 112) and is tracked in the real space of the operating room relative to tracker 112.

The probe tip 120 is tracked in the real space of the operating room relative to tracker 116. With the two trackers 112, 116 in the field of view of optical sensor 102 and the device 122 tracking the current poses thereof, the user interface can be updated to display a representation of the (actual) probe tip 120 in the user interface. In an embodiment, the representation comprises cross hairs 604. The user interface is updated in response to movement of the trackers, for example responsive to movement of the tip toward point H. In an embodiment, additional feedback to the user, such as sound or haptic feedback, responsive to the distance between the tip and the point H can be provided by device 122.

Though not shown, a live view of images may also be presented in the user interface. In an embodiment, the live view images are annotated with a graphic showing the desired second aperture point H on the femur 114. In an embodiment, the live view images are annotated with a graphic highlighting the probe tip 120.

Though not shown, in an embodiment where the computing device is configured to generate a surface model based on the plurality of candidate second aperture points, the user interface can be provided to display the current probe tip (e.g. as a graphical element) and the desired second aperture point with respect to the surface model.

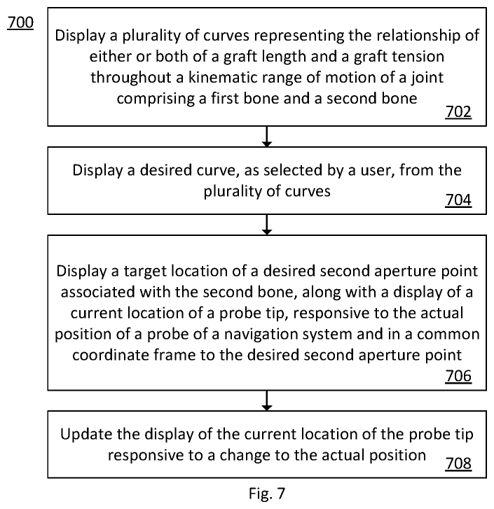

FIG. 7 shows operations 700 of a computer-implemented method that will be understood from the teaching herein. Corresponding user method will be understood but is not illustrated. At 702 the operations display a plurality of curves representing the relationship of either or both of a graft length and a graft tension throughout a kinematic range of motion of a joint comprising a first bone and a second bone. At 702 operations display a desired curve, as selected by a user, from the plurality of curves. For example a user interface is presented having a control to receive input to select one of the curves. Selection of the curve selects the candidate second tunnel aperture point associated with the curve as a desired second tunnel aperture point. At 704 operations display a target location of the desired second aperture point associated with the second bone, along with a display of a current location of a probe tip, responsive to the actual position of a probe of a navigation system and in a common coordinate frame to the desired second aperture point. The actual position is tracked and the current location of the probe tip is updated accordingly. For example, at 706, operations update the display of the current location of the probe tip responsive to a change to the actual position of the probe.

Though FIGS. 4A and 4B show operations 400/450 as having an order of steps 402 to 406/452 to 456 respectively, to registering the kinematic range of motion, registering the first tunnel aperture point 210 and registering the candidate second tunnel aperture points 212, the order of these steps can be varied. For example, the registration of the kinematic range of motion can be performed after the registration of any of the points 210, 212 or between the registration of any of the points 210, 212. Operations can registered some of the second points 212, then the first point 201, then the remaining second points, though it may be preferred such as to minimize time, to proceed in a linear manner.

Moreover, in an embodiment, a second bone tracker (i.e. a dedicated bone tracker) need not be coupled the tibia for example. The probe (e.g. instrument 118) and its tracker 116 can be used to identify the first tunnel aperture point 210 during the range of motion procedure. A separate registration of the first tunnel aperture point need not be performed. The pose pairs determined through such a RoM procedure comprises a pose of the femur tracker and a pose of the probe tracker. The tip 120 of the probe identifies the pose of the first tunnel aperture point 210 during the ROM operations. The poses of the candidate second tunnel aperture points for the range of motion can be determined using the poses of the femur tracker for the range of motion and the spatial relationship to the femur tracker registered for each of the candidate second tunnel aperture points 212. The registration of the spatial relationship for the candidate second tunnel aperture points 212 (in accordance with any of the embodiments described herein) can be performed before or after performing the ROM operations.

In an alternative embodiment to that described at operations 406/456 to register candidate second tunnel aperture points, the second points indicated by probe tip 120 represent a marking of a candidate area for respective tunnel apertures. For example, four (4) such second points can be registered to device 122 defining, at least roughly, the corners of a generally rectangular area on the surface of lateral condyle 214. Computing device 122 can determine a plurality of spaced points in the candidate area using the coordinates of the rectangle. In such an embodiment, the workflow can provide an input control to receive a number of points to space about the rectangle (e.g. 3 or 6 or 8). The larger the number the smaller the distances between each point. Alternatively or in addition, the computing device can use a default number of points (e.g. 8). The user interface can determine the distance (e.g. x, y distances between adjacent points, treating the rectangle as an array) and provide distance or location in text or other form. Poses can be determined for each point defined accordingly. The spacing may be graphically illustrated, for example, which may be an overlay on a live image view. The points may be represented as annotations in live image as noted above. Workflow may be configured to change the number of points and update any user interface accordingly.

In an alternative embodiment to indicating a rectangular area for candidate second tunnel aperture points, the user paints a patch on the bone (e.g. on condyle 214) defining a surface patch (e.g. as a point cloud). A 3D surface more accurately reflects patient anatomy at the patch position relative to a generally rectangular area. Computing device 112 can be configured to determine a plurality of candidate second tunnel aperture points over the surface patch, for example, mapping e.g. a 2D array thereto to space the points associated with the array. The spacing may be graphically illustrated, for example, which may be an overlay on a live image view. The points may be represented as annotations in live image as noted above. Workflow may be configured to change the number of points and update any user interface accordingly.

Though not shown, in an embodiment, system 100 comprises or is communicatively coupled to a robotic system to perform steps of the reconstruction procedure. For example, the robotic system comprises a surgical tool such as a drill (e.g. having one or more components) to effect a tunnel aperture in a bone. In an embodiment, system 100 can be configured to align a trajectory guide with the desired second tunnel aperture point. In an embodiment, the robot system is configured to move the knee through the range of motion procedure, for example, including performing one or more RoMs of a same type and/or of different ROM types. A user may probe the bone for establishing candidate second tunnel aperture points such as in one of the embodiments described.

In an embodiment, the desired curve can be selected automatically (i.e. no need to display a plurality of curves to a user such as for user selection). In an embodiment, guidance to the desired second tunnel aperture need not include a bullseye or other graphic etc. that provides user feedback, particularly where robotic actuation is used to achieve alignment of a desired trajectory to achieve the desired second tunnel aperture.

Though described with reference to single-bundle ACL reconstruction, the methods, systems and apparatus herein can be configured for multi-bundle graft reconstruction such as double-bundle ACL reconstruction. For example, operations may define two respective first tunnel aperture points and candidate second and candidate third tunnel aperture points in respective areas/regions. Graft length changes etc. can be determined respectively over the range of motion using the tracker poses and applicable relationships to the points.

System 100 and operations disclosed show a simple procedure to determine graft length (e.g. changes in length) and related information (tension) through a range of motion. Image based registration where the patient's anatomical structure is registered in system 100 to preoperative patient images or a 3D model, for example, need not be performed. Simplified object tracking can be performed without anatomical registration of the patient's anatomy to system 100.

Practical implementation may include any or all of the features described herein. These and other aspects, features and various combinations may be expressed as methods, apparatus, systems, means for performing functions, program products, and in other ways, combining the features described herein. A number of embodiments have been described. Nevertheless, it will be understood that various modifications can be made without departing from the spirit and scope of the processes and techniques described herein. In addition, other steps can be provided, or steps can be eliminated, from the described process, and other components can be added to, or removed from, the described systems. Accordingly, other embodiments are within the scope of the following claims.

Throughout the description and claims of this specification, the word “comprise”, “contain” and variations of them mean “including but not limited to” and they are not intended to (and do not) exclude other components, integers or steps. Throughout this specification, the singular encompasses the plural unless the context requires otherwise. In particular, where the indefinite article is used, the specification is to be understood as contemplating plurality as well as singularity, unless the context requires otherwise.

Features, integers, characteristics, or groups described in conjunction with a particular aspect, embodiment or example of the invention are to be understood to be applicable to any other aspect, embodiment or example unless incompatible therewith. All of the features disclosed herein (including any accompanying claims, abstract and drawings), and/or all of the steps of any method or process so disclosed, may be combined in any combination, except combinations where at least some of such features and/or steps are mutually exclusive. The invention is not restricted to the details of any foregoing examples or embodiments. The invention extends to any novel one, or any novel combination, of the features disclosed in this specification (including any accompanying claims, abstract and drawings) or to any novel one, or any novel combination, of the steps of any method or process disclosed.

What is claimed is:

1. A navigation system for ligament reconstruction comprising:
a probe with a tip, a tip location of the tip trackable by the navigation system;
a computing device coupled to the navigation system, the computing device comprising at least one processor and memory storing computer-readable instructions executable by the at least one processor to cause the computing device to:
(i) register a kinematic range of motion of a joint, the joint comprising a first bone and a second bone;
(ii) register a first tunnel aperture point for a first bone and a ligament graft in response to the tip location as tracked;
(iii) register a plurality of candidate second tunnel aperture points relative to the second bone in response to respective tip locations as tracked;
(iv) define a plurality of datasets corresponding to the plurality of second tunnel aperture points,
wherein the datasets represent the relationship of either or both of a graft length and a graft tension throughout the kinematic range of motion of the joint;
(v) select a desired dataset from the plurality of datasets; and
(vi) determine a desired second tunnel aperture point on the second bone based on the desired datasets.

2. The system of claim 1, wherein the joint is a knee, the first bone is a tibia, the second bone is a femur and the ligament is an anterior cruciate ligament (ACL).

3. The system of claim 1, wherein the navigation system further comprises one or more of: an optical camera configured for providing optical information for tracking objects, the objects comprising the first bone, the second bone and the probe; a first bone tracker configured for coupling to the first bone for tracking the first bone; or a second bone tracker configured for coupling to the second bone for tracking the second bone.

4. The system of claim 3, wherein the kinematic range of motion is based on poses of the first bone tracker and/or the second bone tracker during a range of motion.

5. The system of claim 1, wherein the computer-readable instructions are executable by the at least one processor to cause the computing device to provide a user interface to display the plurality of datasets.

6. The system of claim 5, wherein to select the desired dataset comprises receiving user input to select one of the datasets as the desired dataset.

7. The system of claim 1, wherein the computer-readable instructions are executable by the at least one processor to cause the computing device to provide a user interface to display the plurality of datasets as respective curves.

8. The system of claim 1, wherein the computer-readable instructions are executable by the at least one processor to select the desired dataset automatically without user selection.

9. The system of claim 1, wherein the computer-readable instructions are executable by the at least one processor to cause the computing device to provide a user interface to guide a user to identify the desired second tunnel aperture point on the second bone using the tip of the probe.

10. The system of claim 9, wherein the computer-readable instructions are executable by the at least one processor to cause the computing device to generate a surface model associated to the plurality of candidate second tunnel aperture points, and wherein the user interface is configured to display the probe tip as tracked and the desired second tunnel aperture point with respect to the surface model.

11. The system of claim 1, wherein to register the first tunnel aperture point defines a spatial relationship between the first tunnel aperture point identified by the tip of the probe and a pose of a first tracker coupled to the first bone, and wherein the pose of the first tunnel aperture point is determinable from tracking the first tracker.

12. The system of claim 1, wherein the computer-readable instructions are executable by the at least one processor to cause the computing device to register the first tunnel aperture point as the computing device registers the kinematic range of motion of the joint, the computing device tracking each of the probe tip and the second bone during the range of motion.

13. The system of claim 1, wherein the computer-readable instructions are executable by the at least one processor to cause the computing device to register the plurality of candidate second tunnel aperture points by receiving the respective tip locations as either: A) points marking an area of the second bone or B) a painting of a surface patch of the second bone; the computer-readable instructions executable by the at least one processor to cause the computing device to determine the candidate second tunnel aperture points within the area or surface patch. The system of claim 1, wherein the navigation system further comprises or is communicatively coupled to a robotic system, and wherein the computer-readable instructions are executable by the at least one processor to cause the computing device to align a trajectory guide of the robotic system with the desired second aperture point.

14. A computer-implemented method comprising:
registering a kinematic range of motion of a joint, the joint comprising a first bone and a second bone;
registering a first tunnel aperture point for a first bone and a ligament graft in response to a tip location of a probe as tracked;
registering a plurality of candidate second tunnel aperture points relative to the second bone in response to respective tip locations of the probe as tracked;
defining a plurality of datasets corresponding to the plurality of second tunnel aperture points,
wherein the datasets represent the relationship of either or both of a graft length and a graft tension throughout the kinematic range of motion of the joint;
selecting a desired dataset from the plurality of datasets; and
determining a desired second tunnel aperture point on the second bone based on the desired dataset.

15. A method to perform a ligament reconstruction comprising:
registering to a navigation system a kinematic range of motion of a joint comprising a first bone and a second bone;
registering to the navigation system a first tunnel aperture point for the first bone and a ligament graft using a probe having a tip trackable by the navigation system;
registering to the navigation system a plurality of candidate second tunnel aperture points relative to the second bone using the probe as tracked;
selecting a desired datasets from a plurality of curves representing the relationship of either or both of a graft length and a graft tension throughout the kinematic range of motion of the joint, wherein the plurality of datasets are presented by a user interface; and
identifying a desired second tunnel aperture point on the second bone based on the desired dataset as selected.

16. The method of claim 15, wherein the navigation system is configured to track the first bone and the second bone using an optical sensor comprising a camera.

17. The method of claim 15, wherein the joint is a knee, the ligament graft is an anterior cruciate ligament (ACL), the first bone is a tibia, and the second bone is a femur.

18. The method of claim 15, further comprising receiving guidance from the navigation system for locating, using the tip of the probe, the desired second tunnel aperture point on the second bone.

19. A computer-implemented method comprising:
displaying a plurality of datasets representing the relationship of either or both of a graft length and a graft tension throughout a kinematic range of motion of a joint comprising a first bone and a second bone;
receiving input selecting a desired dataset from the plurality of datasets; and
displaying a target location of a desired second tunnel aperture point associated with the second bone, along with a display of a current location of a probe tip, responsive to the actual position of a probe of a navigation system and in a common coordinate frame to the desired second aperture point.

20. The method of claim 19, wherein each of the plurality of datasets is associated with one of a plurality of second tunnel aperture points on the second bone, and wherein the desired second tunnel aperture point is determined in accordance with the association.
